(19) United States
(12) Patent Application Publication
(10) Pub. No.: US 2026/0090704 A1
(43) Pub. Date: Apr. 2, 2026

(54) MICROMOTOR AND OPTICAL ARRANGEMENT FOR FAST CIRCULAR SCANNING OF LIGHT BEAMS IN SMALL DIAMETER FLEXIBLE CATHETERS

(71) Applicant: The General Hospital Corporation, Boston, MA (US)

(72) Inventors: Ginger Schmidt, Boston, MA (US); Brett E. Bouma, Boston, MA (US); Milen Shishkov, Boston, MA (US); Néstor Uribe-Patarroyo, Boston, MA (US)

(21) Appl. No.: 19/110,542

(22) PCT Filed: Sep. 19, 2023
(86) PCT No.: PCT/US2023/074596

§ 371 Date: Mar. 11, 2025

Related U.S. Application Data
(60) Provisional application No. 63/376,423, filed on Sep. 20, 2022.

(51) Int. Cl. A61B 1/00; A61B 8/12; A61M 25/01

(52) U.S. Cl. CPC A61B 1/00158; A61B 1/00096; A61B 1/00165; A61B 8/12; A61M 25/0127

(57) ABSTRACT

The disclosure presents a shaftless, brushless motor for rotating any optical or sensing element (e.g., a lens or mirror) at a distal end of a waveguide that also delivers electromagnetic radiation (e.g., light). Herein, the waveguide itself functions as the axle on which rotating components rotate, thereby avoiding blind spots and overcoming limitations in existing “micromotors”. The shaftless, distally driven motor significantly reduces motor size, while extremely small inertial loads and bearing sizes allow for high pitch cylindrical scanning with longitudinal velocity uniformity.

### CROSS REFERENCE TO RELATED APPLICATIONS

The present application is based on, claims priority to, and incorporates herein by reference in its entirety for all purposes, U.S. Provisional Application Ser. No. 63/376,423, filed Sep. 20, 2022.

### STATEMENT OF GOVERNMENT SUPPORT

This invention was made with government support under P41EB015903 awarded by NIH/NIBIB. The U.S. government has certain rights in the invention.

### BACKGROUND

Considerable effort has been devoted towards developing miniaturized high-speed scanning elements (e.g., optical mirror or sensors) for catheter or endoscopic imaging or sensing. For instance, when located at the tip of an optical fiber that delivers light, rotating optical elements allow for radial rotational scanning and sampling, such as may be required for biomedical imaging or general testing and measurement. To increase sampling speeds, it is desirable for such motors to rotate as fast and uniformly as possible while maintaining functional integrity of the entire system.

Existing motor drive units rotate components through the length of an entire catheter sheath with a conventional motor proximal and outside to the device. An optical rotary junction transmits signals through a rotating interface outside and up to many meters away from the desired field of view at the distal tip of the catheter. Since all components rotate within the non-rotating sheath, friction between rotating and non-rotating components severely limits maximum rotational speeds to 100 Hz, while simultaneously leading to undesirable non-uniform rotational distortion.

One solution utilizes a miniaturized brushless motor with a shaft at the very distal tip. The motor faces backwards towards the waveguide, allowing rotating components to interface directly with incoming electromagnetic radiation, rather than having to rotate the waveguide itself through the length of the entire device. This avoids significant limitations introduced by friction between rotating components and the sheath, allowing for rotational speeds up to 2 KHz. However, for the backwards facing motor to be powered, wires must pass through the field of view, creating unavoidable blind spots in the rotational field of view. Previous motor architectures are also difficult to further miniaturize and are too costly for implementation in disposable medical equipment (e.g., intravascular imaging catheters).

Accordingly, there is a need to overcome one or more of the above-described deficiencies.

### SUMMARY

The present disclosure provides systems and methods that overcome one or more of the aforementioned drawbacks via a shaft-less, brushless synchronous motor configured to rotate an optical element (e.g., a lens or mirror) at a distal end of a waveguide that delivers electromagnetic radiation (e.g., light). For imaging applications, the fiber tip, and/or the subsequent optical element associated with the fiber tip, is configured to provide precise focusing of the emitted light. The waveguide additionally provides a bearing support of the rotor of the motor.

In one aspect, an imaging system is disclosed. The imaging system includes a power source, a catheter having a proximal end and a distal end disposed and defining a lumen therebetween, and an imaging assembly having a proximal portion and a distal portion disposed within the lumen adjacent to the distal end of the catheter. The imaging assembly further includes a waveguide centered in the imaging assembly and extending the length of the catheter, at least one permanent magnet positioned in the proximal portion of the imaging assembly and disposed radially around the waveguide, and an optical element coupled to an outer surface of the at least one permanent magnet. The optical element is disposed beyond the distal portion of the imaging assembly and into the distal end of the catheter. The imaging assembly further includes one or more electromagnetic coils positioned in the proximal portion of the imaging assembly and radially outward from the at least one permanent magnet. The electromagnetic coils are in electrical communication with the power source.

In certain embodiments of the imaging system, the at least one permanent magnet may include a ring magnet.

In certain embodiments of the imaging system, the waveguide may be one or more optical fibers and may include one or more radial protrusions. In one embodiment of the imaging system, the one or more radial protrusions may be formed in the waveguide. In other embodiments of the imaging system, the radial protrusions may include bearings radially positioned on the waveguide.

In certain embodiments of the imaging system, the one or more radial protrusions may abut the at least one permanent magnet.

In certain embodiments of the imaging system, the distal tip of the waveguide may include a lens.

In certain embodiments of the imaging system, the optical element may include one or more mirrors or lenses.

In certain embodiments of the imaging system, an extension may couple the optical element to the outer surface of the one or more permanent magnet. In one aspect, the extension may be a tube. The extension may include an opening in at least a portion thereof. In one embodiment of the imaging system, the waveguide may be configured to transmit light toward the one or more mirrors whereupon the light may be reflected through the opening in the extension.

In certain embodiments of the imaging system, the electromagnetic coils may be in electrical communication with the power source via one or more wires. In other embodiments of the imaging system, the electromagnetic coils may generate a magnetic field when a current is supplied by the power source. In another embodiment of the imaging system, a back iron may be positioned radially between the one or more electromagnetic coils and an inner surface of the catheter in the proximal portion of the imaging assembly.

In certain embodiments of the imaging system, the at least one permanent magnet and optical element may rotate based on a strength and a frequency of the magnetic field.

In certain embodiments of the imaging system, a support bearing may be coupled to the extension and abut a distal end of the one or more electromagnetic coils.

In certain embodiments of the imaging system, a spring may be positioned proximally to the at least one permanent magnet in the imaging assembly and oriented such that the waveguide passes through the spring.

In certain embodiments of the imaging system, an ultrasound transducer may be coupled to the extension.

In another aspect, an imaging assembly having a proximal portion and a distal portion disposed with a distal end of a catheter is described. The imaging assembly includes a waveguide centered in the imaging assembly and extending the length of the catheter, at least one permanent magnet positioned in the proximal portion of the imaging assembly and disposed radially around the waveguide, and an optical element coupled to an outer surface of the at least one permanent magnet. The optical element is disposed beyond the distal portion of the imaging assembly and into the distal end of the catheter. The imaging assembly further includes one or more electromagnetic coils positioned in the proximal portion of the imaging assembly and radially outward from the at least one permanent magnet.

In certain embodiments of the imaging assembly, the at least one permanent magnet may include a ring magnet.

In certain embodiments of the imaging assembly, the waveguide may be one or more optical fibers and may include one or more radial protrusions. In one embodiment of the imaging assembly, the one or more radial protrusions may be formed in the waveguide. In other embodiments of the imaging assembly, the radial protrusions may include bearings radially positioned on the waveguide.

In certain embodiments of the imaging assembly, the one or more radial protrusions may abut the at least one permanent magnet.

In certain embodiments of the imaging assembly, the distal tip of the waveguide may include a lens.

In certain embodiments of the imaging assembly, the optical element may include one or more mirrors or lenses.

In certain embodiments of the imaging assembly, an extension may couple the optical element to the outer surface of the one or more permanent magnet. In one aspect, the extension is a tube. The extension may include an opening in at least a portion thereof. In one embodiment of the imaging assembly, the waveguide may be configured to transmit light toward the one or more mirrors whereupon the light may be reflected through the opening in the extension.

In certain embodiments of the imaging assembly, the electromagnetic coils may generate a magnetic field when a current is supplied by the power source. In another embodiment of the imaging assembly, a back iron may be positioned radially between the one or more electromagnetic coils and an inner surface of the catheter in the proximal portion of the imaging assembly.

In certain embodiments of the imaging assembly, the at least one permanent magnet and optical element may rotate based on a strength and a frequency of the magnetic field.

In certain embodiments of the imaging assembly, a support bearing may be coupled to the extension and abut a distal end of the one or more electromagnetic coils.

In certain embodiments of the imaging assembly, a spring may be positioned proximally to the at least one permanent magnet in the imaging assembly and oriented such that the waveguide passes through the spring.

In certain embodiments of the imaging assembly, an ultrasound transducer may be coupled to the extension.

In another aspect, an imaging method is disclosed. The method includes providing an imaging system. The imaging system includes a waveguide centered in the imaging assembly and extending the length of the catheter, at least one permanent magnet positioned in the proximal portion of the imaging assembly and disposed radially around the waveguide, and an optical element coupled to an outer surface of the at least one permanent magnet. The optical element is disposed beyond the distal portion of the imaging assembly and into the distal end of the catheter. The imaging assembly further includes one or more electromagnetic coils positioned in the proximal portion of the imaging assembly and radially outward from the at least one permanent magnet. The method further includes providing a current to the one or more electromagnetic coils to generate a magnetic field, transmitting one or more beams of light through the waveguide towards the optical element, receiving one or more reflected beams of light from the optical element, and generating an image from the one or more reflected beams using a processor.

In certain embodiments of the imaging method, the optical element may include one or more mirrors or lenses.

In certain embodiments of the imaging method, an extension may couple the optical element to the outer surface of the one or more permanent magnet. In one aspect, the extension may be a tube. The extension may include an opening in at least a portion thereof. In various embodiments of the imaging method, light may be transmitted using the waveguide toward the one or more mirrors such that the light is reflected through the opening in the extension.

In certain embodiments of the imaging method, the electromagnetic coils may be electrically connected with the power source via one or more wires. In particular embodiments of the imaging method, a magnetic field may be generated using the electromagnetic coils by transmitting a current from the power source. In various embodiments of the imaging method, the at least one permanent magnet and optical element may be rotated based on a strength and a frequency of the magnetic field.

In certain embodiments of the imaging method, a back iron may be provided which is positioned radially between the one or more electromagnetic coils and an inner surface of the catheter in the proximal portion of the imaging assembly.

In certain embodiments of the imaging method, a support bearing may be provided which is coupled to the extension and abutting a distal end of the one or more electromagnetic coils.

In certain embodiments of the imaging method, a spring may be provided which is positioned proximal to the at least one permanent magnet in the imaging assembly and oriented such that the waveguide passes through the spring.

In certain embodiments of the imaging method, ultrasound energy may be transmitted using an ultrasound transducer couped to the extension. These aspects are nonlimiting. Other aspects and features of the systems and methods described herein will be provided below.

### BRIEF DESCRIPTION OF THE DRAWINGS

The foregoing features of embodiments will be more readily understood by reference to the following detailed description, taken with reference to the accompanying drawings, in which:

FIG. 1 is a schematic of an imaging system, according to aspects of the present disclosure.

FIG. 2A is a schematic of the catheter and imaging assembly according to aspects of the present disclosure.

FIG. 2B is a forward-facing view of the imaging assembly shown in FIG. 2A FIG. 2C is a schematic of the radial scanning imaging assembly, according to aspects of the present disclosure.

FIG. 2D is a schematic of the forward scanning imaging assembly, according to aspects of the present disclosure.

FIG. 3 is a schematic of a flexible circuit electromagnetic coil, according to aspects of the present disclosure.

FIG. 4A is a schematic of an imaging assembly with offset electromagnetic coils from a magnet's magnetic field, according to an aspect of the present disclosure.

FIG. 4B is a schematic of the imaging assembly of FIG. 4A recentered in the magnet's magnetic field, according to aspects of the present disclosure.

FIG. 5A is an embodiment of bulge bearings of on the waveguide, according to aspect of the present disclosure.

FIG. 5B is another embodiment of bulge bearings of on the waveguide, according to aspect of the present disclosure.

FIG. 5C is another embodiment of bulge bearings of on the waveguide, according to aspect of the present disclosure.

FIG. 5D is another embodiment of bearings of on the waveguide, according to aspect of the present disclosure.

FIG. 5E is another embodiment of bearings of on the waveguide, according to aspect of the present disclosure.

FIG. 6A is a schematic of a tube supporting an ultrasound transducer, according to aspects of the present disclosure.

FIG. 6B is a schematic of a tube supporting two optical elements in series, according to aspects of the present disclosure.

FIG. 6C is a schematic of a tube supporting an optical element through which two light beams are transmitted, according to aspects of the present disclosure.

FIG. 7 is a schematic of the imaging assembly configured for pull back scanning, according to aspects of the present disclosure.

FIG. 8 is a flowchart of a method of imaging, according to aspects of the present disclosure.

### DETAILED DESCRIPTION

A shaftless, brushless motor is disclosed to rotate an optical or sensing element (e.g., mirror, lens, prism, second waveguide, etc.) directly adjacent to (e.g., distal to or in front of) the tip of a waveguide that delivers electromagnetic radiation (e.g., light), acoustic energy, or ultrasound energy. The waveguide itself also functions as the axle on which rotating components of the motor rotate. This unique feature contrasts with prior designs wherein the motor is distal to the waveguide tip and generally oriented in a backward-facing configuration. Such prior designs require electrical wires to pass through the beam emitted by the waveguide, thereby resulting in blind spots. In addition to overcoming the prior limitation of blind spots in the imaging or sensing capabilities of the device, the present invention additionally permits further reductions in motor size (e.g., in diameter and/or length) and extremely small inertial loads and bearing sizes, which allow for operational speeds exceeding those of conventional catheter designs. Specifically, operational speeds exceeding 100 revolutions per second may be provided. For imaging applications wherein motion artifacts may require mitigation or wherein the monitoring of rapid dynamic processes is of interest, operation speeds of the motors of the present invention may exceed 3,000 revolutions per second. In one embodiment, the disclosed motor has operation speeds between 3,000 revolutions per second and 6,000,000 revolutions per second. A further advantage of the present invention is that it may be less expensive and less complex to manufacture, which facilitates using the device in a disposable catheter to provide better reliability and/or improved sterility.

The unique design features of the present invention permit the fabrication of motors having outside diameters between 0.15 mm and 3.00 mm. In one embodiment, the motor outer diameter is less than 1 mm. For intravascular imaging, the present invention enables the fabrication of motors having an outside diameter less than 0.7 mm diameter; for neuro-vascular imaging, the present invention enables the fabrication of motors having a diameter less than 0.5 mm diameter.

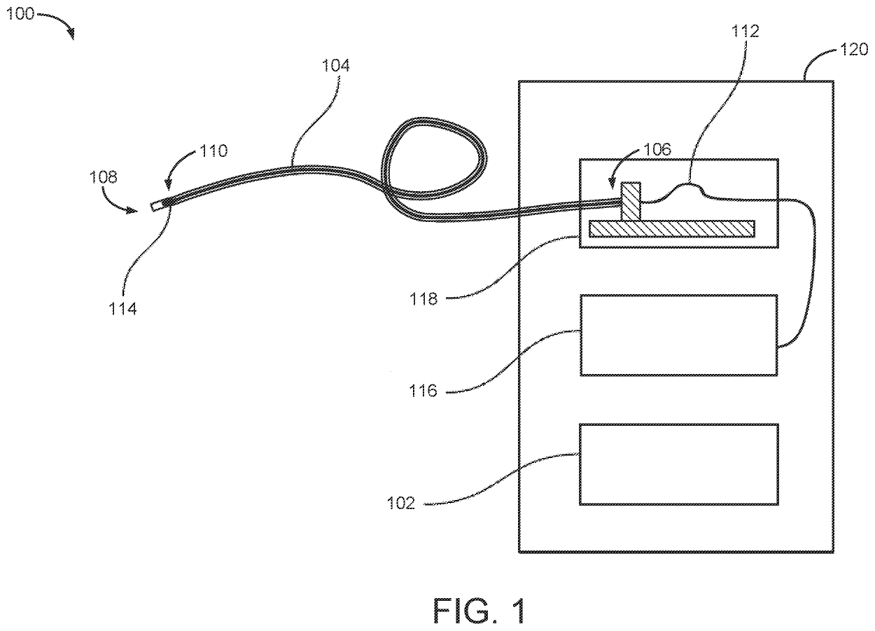

In a non-limiting example, FIG. 1 is an exemplary imaging system 100 according to certain embodiments. The imaging system 100 includes a power supply 102. In a non-limiting example, the power supply 102 may be at least one direct current (DC, such as a battery) voltage source or alternating current (AC) voltage source (such as a wall outlet or other time-varying power supply). The system 100 may further include a catheter 104 comprising a proximal end 106 and a distal end 108. In a non-limiting example, the catheter 104 is made of flexible polymeric materials such as polyamide, polyurethane, or polytetrafluoroethylene. Alternatively, the catheter may be made of a braided copper wire tube. The catheter 104 further defines a lumen between the proximal end 106 and distal end 108.

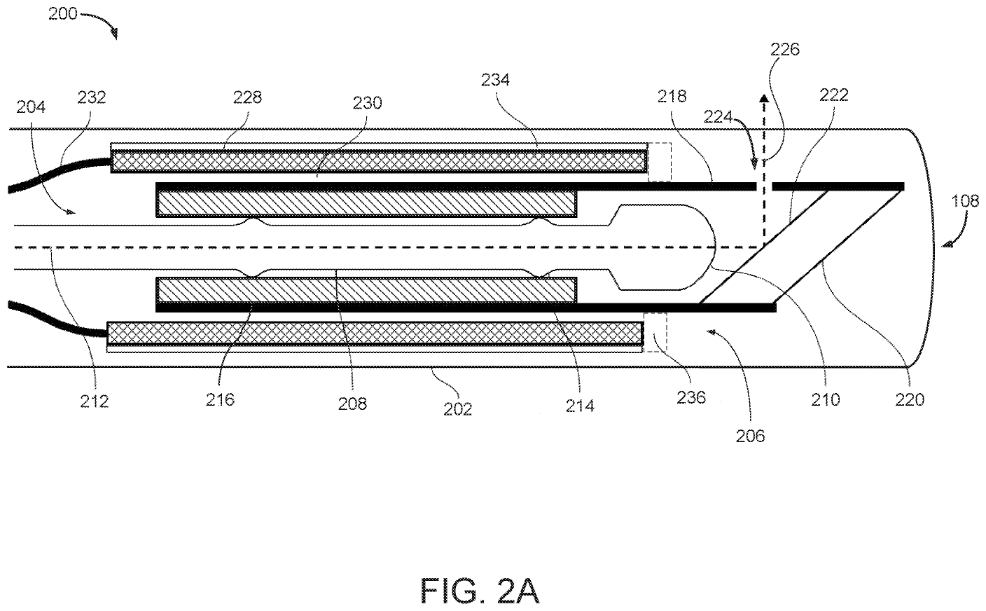

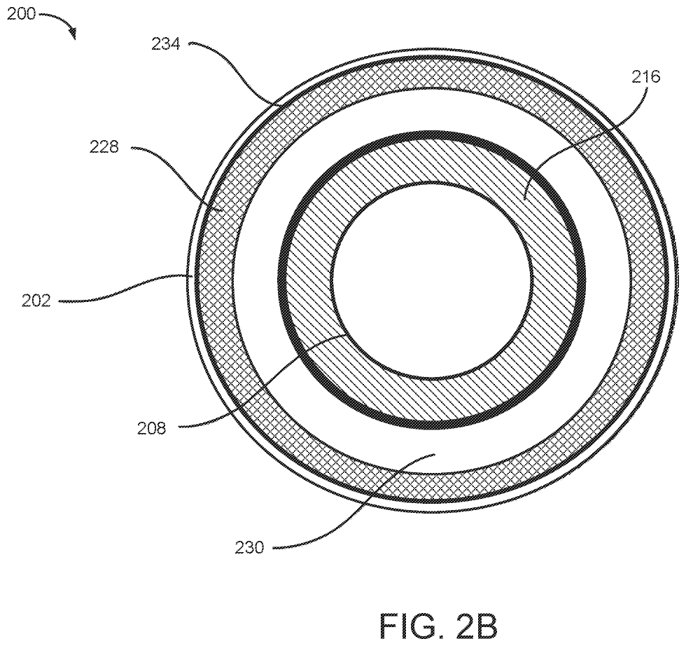

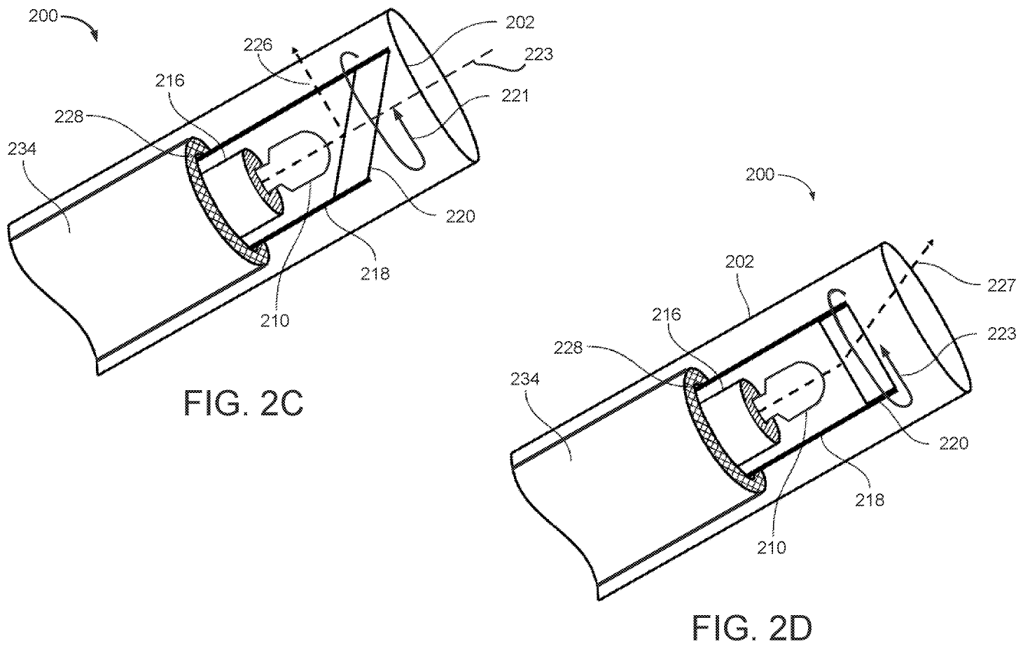

In a non-limiting example, the catheter includes an imaging assembly 110 disposed within the lumen and adjacent to the distal end 108. In one example, the imaging assembly 110 includes a micromotor 114. The micromotor 114 is described in further detail in FIGS. 2A-2D. In a non-limiting example, the micromotor 114 is in electrical communication with the power supply 102. In a non-limiting example, the electrical communication may be a wired connection. Alternatively, the electrical communication may be wireless between the micromotor 114 and power supply 102. In other embodiments, other forms of energy such as optical energy may be used to transmit power which is then converted to electricity.

In a non-limiting example, the imaging system 100 further includes an optical unit 116 including one or more light sources and source detectors. For example, the light source may be or include one or more of an optical coherence tomography (OCT), ultrasound imaging, and/or fluorescence imaging source.

In a non-limiting example, a pull-back device 118 is included in the imaging system 100 for pulling back the catheter 104 through a vessel during imaging.

In a non-limiting example, the power source 102, optical unit 116, and pull-back device 118 are individual units or may be integrated into a single control unit 120. For example, the control unit 120 may be a processor.

Referring now to FIGS. 2A-2D, a distal end of the catheter 104 is shown providing further detail of the imaging assembly 200 and micromotor. In a non-limiting example, the imaging assembly 200 is positioned adjacent to the distal end 108 within the catheter sheath 202.

In a non-limiting example, the imaging assembly 200 defines a proximal portion 204 and a distal portion 206. In this non-limiting example, the imaging assembly 200 includes a waveguide 208 centered in the catheter sheath 202. For example, the waveguide 208 may be one or more optical fibers. The waveguide 208 further comprises a distal tip 210. The distal tip 210 may be or include a lens for focusing a light beam 212 transmitted through the waveguide 208.

In a non-limiting example, the waveguide 208 further includes one or more protrusions or bearing bulges 214 protruding radially from the waveguide to maintain spacing and/or facilitate rotation between the waveguide 208 and the magnet 216. In one example, the bearing bulges 214 may be formed directly into the waveguide itself by forming larger diameter regions in two or more locations through splicing or heated shaping of the optical fiber. Alternatively, the bearing bulges 214 may be bearings positioned around the waveguide 208 made of a separate material from the waveguide 208, such as ruby, rubber, or epoxy.

In a non-limiting example, the rotor of micromotor 114 may be supported by the waveguide itself. To reduce friction between rotating components and the waveguide, the bearing bulges 214 may provide circumferential contact points with a smaller surface area. The motor's rotor thus slides over the “bulges” but only contacts the waveguide through those two or more points, thus reducing friction. Alternatively, forming an uneven matte surface on the outer diameter of the waveguide would also reduce the surface area that is in contact between the waveguide and adjacent rotating elements.

In a non-limiting embodiment, the rotor of the micromotor 114 is or includes at least one permanent magnet 216. The permanent magnet may be a ring magnet as illustrated in FIGS. 2A-2D. However, any shape and/or number of such magnet(s) may be used provided they accommodate a central opening through which the waveguide 112 passes. In one example, the magnet 216 is a diametrically magnetized permanent magnet with one or more pole pairs, where the use of multiple poles can increase motor speed, torque, and/or efficiency. The magnet 216 may be, for example, sintered, electrical discharge machined, or metal 3D printed. The magnet 216 material may be iron neodymium but may also be some other magnetic material such as iron, nickel, or cobalt. In a non-limiting example, the motor's rotor should also be radially and axially balanced to prevent undesirable gyration or wobbling and to enable high speed rotation.

The magnet 216 is positioned in the proximal portion 204 of the imaging assembly 200 and disposed radially around the waveguide 208. Any aforementioned embodiments of bulges 214 or bearings could be located either fully within the hollow rotating elements, pressed up outside and at each end of rotating components to prevent axial movement, or some combination of both.

In one embodiment, the magnetic rotor may be attached to a distal optical component that is operative to redirect a beam emitted from the axially located waveguide. In this embodiment, it is desirable that the combined structure comprising the rotor, the optical component, and any associated mechanical mounting element be radially and axially balanced. In such embodiments, it may be preferable for the center of mass of one component to be configured to offset any non-axial portion of the center of mass of another component or components. As shown in FIG. 2A, an extension 218 is attached at a proximal end to the outer radial face of the magnet 216 while the distal end of the extension 218 has an optical element 220 coupled thereto. In a non-limiting example, the extension 218 is, but is not limited to, a tube, arm, or bracket. In embodiments where the extension 218 is a tube (such as in FIGS. 2A-2D), the cross section may be circular. In another example, the cross section of the tube extension 218 may be any shape that conforms to the outer surface of the at least one permanent magnet. In one example, the tube 218 may be transparent to the spectrum of the beam emitted from the waveguide 208. Many transparent materials are possible, but in the case of an optical beam, they may include polyimide, acrylic, glass, crystalline material, or any suitable plastic.

In embodiments of the present invention, the permanent magnet 216 may be connected to additional elements 220 that interact with a light beam 212 transmitted through and emitted from the waveguide 208. Such elements may be configured to generate a uniformly circular focal spot or any other arbitrarily engineered beam profile or profiles. Certain embodiments may include elements that are structured to compensate for a particular aberration of the emitted beam or to (pre)compensate for aberrations that may arise from subsequent transmission of the beam through additional elements or structures or samples.

In a non-limiting example, the optical element 220 at the other end of the tube 218 includes a prism or prisms, multiple mirrors, partially reflecting mirrors, any diffractive and/or refractive optical elements such as lenses, or other generic components used for imaging or sensing, such as an ultrasound transducer. Components may also be mounted at any angle to allow for both transverse and forward or backwards conical scanning, such as with mirrors in order to avoid Fresnel reflections that may arise from any enclosing material, structure, or sample. All such elements may also support multiple beams, such as for fluorescence imaging, which requires at least one illumination beam and one detection beam, or as in the case of photoacoustic imaging wherein optical energy is delivered to a sample and ultrasound energy is collected from the sample.

In one example, the mirror(s) are configured to deflect the light beam path based on an angular position of the surface of the mirror(s) relative to the transmitted light beam 212. Alternatively, the angle of the mirror 220 provides an astigmatism correction 222. In this example, the imaging assembly 200 is utilized for radial scanning (FIG. 2C) as the optical element 220 rotates 221 around an axis of rotation 223. In a non-limiting example, the tube 218 includes at least a partial opening 224 adjacent to the optical element 220 configured to allow the reflected light beam or beams 226 to pass directly from the rotating element to a location radially displaced from the axis of the waveguide (FIG. 2A). In one embodiment, the tube 218 may support a mirror that simply redirects and focuses the emitted beam for imaging.

Alternatively, the beam may pass through one or more diffractive or refractive optical elements 220 such as lenses for forward scanning (FIG. 2D) as the optical element rotates around the axis of rotation 223. In the non-limiting example for forward scanning, the tube 218 does not include at least a partial opening on the longitudinal surface of the tube 218 since the light beam 212 enters and exits the diffractive or refractive optical element 220 as a diffracted or refracted light beam 227.

In a non-limiting example, the micromotor 114 further includes one or more electromagnetic coils 228 positioned in the proximal portion 204 of the imaging assembly 200 and radially outward from the tube 218 coupled to the permanent magnet 216 (FIG. 2A). In a non-limiting example, the electromagnetic coils 228 are spaced apart from the radially inward tube 218 by an air gap 230 to reduce friction and to allow the rotor to freely rotate (FIG. 2A). Alternatively, the airgap may be filled with a liquid lubricant and/or cooling solution, such as saline or oil. The one or more electromagnetic coils 228 are configured to carry a time-varying current circumferential to the permanent magnet 216, thereby changing the external magnetic field to cause the permanent magnet 216 to rotate. In a non-limiting example, the one or more electromagnetic coils 228 are in electrical communication with the power source 102 via one or more wires 232 (FIG. 2A). Although the coils 228 are generally shown herein as generating external magnetic fields to cause the permanent magnet 216 to rotate, in some non-limiting examples the changing electromagnetic fields that cause the magnet to rotate may also be generated outside of the catheter or device, such as outside the body for biomedical applications.

Optionally, in some non-limiting examples a back iron 234 may surround the electromagnetic coils 228 to provide a more efficient return path of flux between electromagnets within the rotor, thus increasing motor torque (FIGS. 3A-2D, 4A, 4B). However, a back-ironless design may also be used to allow for a larger magnet, which would also increase torque (e.g., FIG. 2A). Suitable ferrous materials for the back iron 234 may be provided in the form of a sleeve surrounding the coil or they may be provided as a layer or coating to the inner or outer surface of the catheter sheath 202.

In another non-limiting example, the imaging assembly 200 includes one or more tube support bearings 236 between the outer surface of the tube 218 and the distal surface of the electromagnetic coils 228 to radially and axially balance the rotating elements, including one or more of the ring magnet 216, the tube 218, and/or the optical element 220.

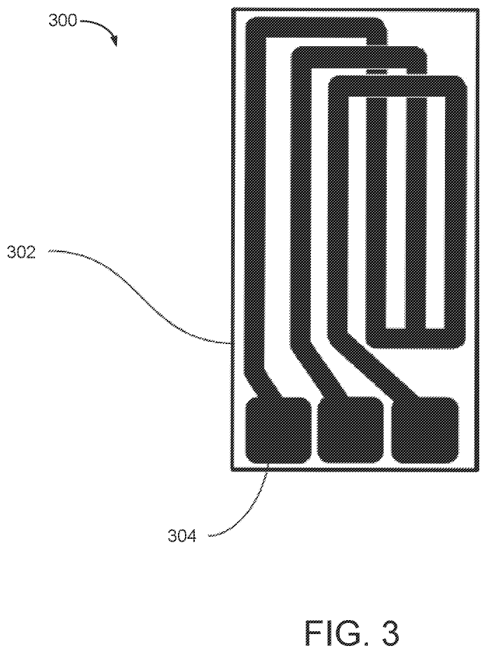

Referring back to the one or more electromagnetic coils 228, in a non-limiting example the coils may be made from flat, flexible printed circuit materials 302 that are rolled into a cylinder, or in another non-limiting example may be directly printed onto a cylindrically shaped circuit material through photolithography (FIG. 3). Suitable circuits may include one or more layers and may have one or more turns 304 per layer. Each turn is coupled to a solder pad. Although FIG. 3 illustrates an unfurled 3-phase circuit electromagnetic coil (where the long axis of the motor is vertical in this view), the number of phases may be less than three or more than three. Alternatively, in other non-limiting examples the electromagnetic coils 228 may also be wound directly from thin wires and shaped around the magnet. In further non-limiting examples, the coils may also be 3D printed with metals or laser sintered onto a substrate such as glass.

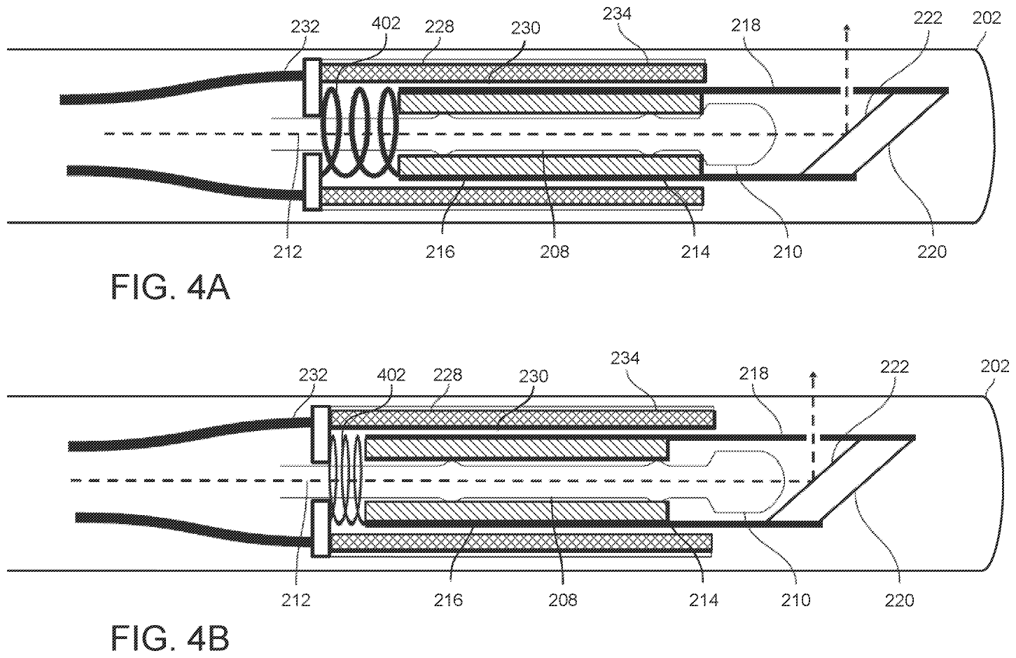

For any of the above embodiment of the coils, they may be wired in a three-phase delta, star, or wye configuration. For magnets that have more than one pole pair, coils may correspondingly also have more than three phases. Referring to FIGS. 4A-4B, in an alternative embodiment, the one or more electromagnetic coils 228 may be placed so that they are offset relative to the axial and/or longitudinal center of the magnetic field of the permanent magnet 216, so they may generate an intentional longitudinal force on the magnet. To allow for movement in response to such a force, while maintaining its position otherwise, the magnet may be held in by a spring 402. The strength of the magnetic field of the coils 228 can be varied by varying the amplitude of the current sent to the coils. When the current to the coils 228 increases, the field strength is increased which then causes the permanent magnets 216 to become centered within the coils 228, with the permanent magnets 216 causing the tube 218 to compress the spring 402. When the current to the coils is reduced, thereby reducing the field strength of the coils 228, the spring 402 then pushes the tube 218 in a distal direction. This varying of field strength would then generate longitudinal motion that can be harness for any purpose, including longitudinal scanning and refocusing (since this also varies the distance between the distal tip lens 210 and the optical element 220).

Longitudinal motion of the micromotor and/or imaging assembly within the catheter sheath may alternatively be provided by providing tension to the wires that may be attached to the coil of the motor. In another exemplary embodiment, said wires may be embedded within the lumen of a thin-walled tubing that may comprise an additional layer between the waveguide and the catheter sheath. In this embodiment, tension or actuation of the thin-walled tubing layer may enable longitudinal motion of the motor and/or associated beam delivery components.

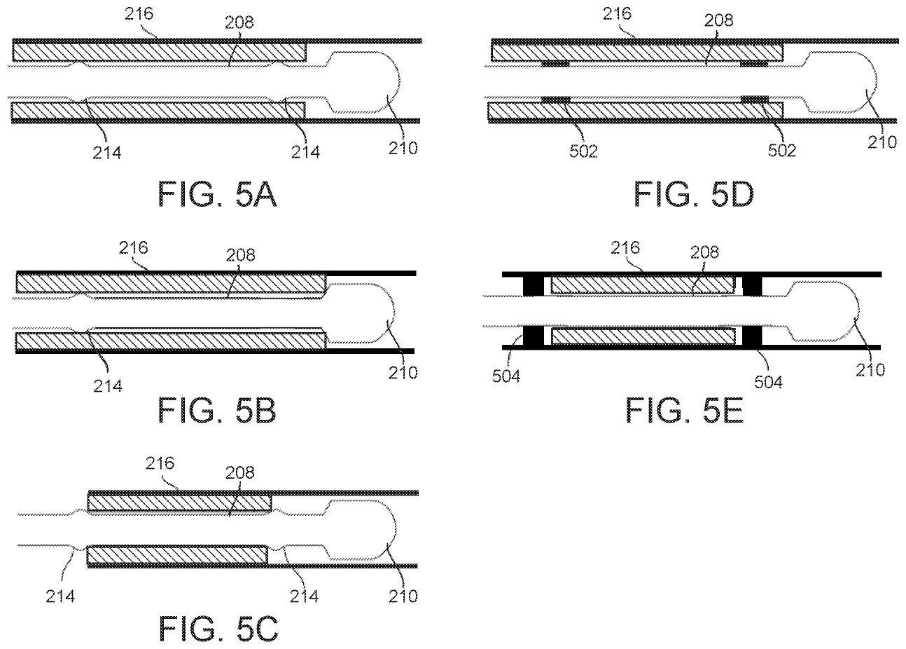

FIGS. 5A-5E show additional configurations of the bearing bulges between the waveguide and the ring magnet. In the non-limiting example of FIG. 5A, the waveguide 208 has two bearing bulges 214 formed in the waveguide 208 itself. It is noted that, although FIG. 5A shows two bearing bulges 214, any number of bearing bulges 214 may be included. The two bearing bulges 214 abut the permanent magnet 216 and prevent the entire surface of the permanent magnet 216 from coming into contact with the surface of the waveguide 208, apart from where the bulges 214 are in contact with the permanent magnet 216.

In another non-limiting embodiment shown in FIG. 5B, the waveguide includes a single bearing bulge 214 formed in the waveguide 208 to prevent contact between the waveguide 208 and the permanent magnet 216. Further, in this configuration, the distal tip 210 of the waveguide 208 has a larger radius than the length of the waveguide 208. This larger radius distal tip 210 provides a distal contact point to prevent translation into the distal end of the catheter. In another non-limiting example, this larger radius distal tip 210 may also be included in the embodiment of FIG. 5A. Including at least two points of contact between the waveguide 208 and the permanent magnet 216 helps to stabilize the components relative to one another.

In another non-limiting example shown in FIG. 5C, the waveguide 208 includes at least two bulge bearings 214 that are used to both prevent axial translation of the permanent magnet 216 and to limit the amount of contact between the permanent magnet 216 and the waveguide 208. In yet another non-limiting example, this configuration may also be applied to the embodiment of FIG. 5B, wherein the permanent magnet 216 is positioned only between the bearing bulge 214 and the larger radius distal tip 210.

In alternative embodiments, the bearing bulge 214 of the above embodiments may be replaced with a separate bearing material disposed radially around the waveguide 208 (as opposed to having the bearing bulges 214 being formed from the waveguide material itself). As previously described, the materials for the applied bearings 502 may be, but are not limited to, ruby, rubber, or epoxy.

In a non-limiting example of FIG. 5D, one or more separate bearings 502 are radially disposed around the waveguide and provide the contact points with the permanent magnet 216 to reduce friction.

In another non-limiting example shown in FIG. 5E, one or more separate bearings 504 are provided between the waveguide 208 and the tube 218, further providing spacing between the permanent magnet 216 and the waveguide 208.

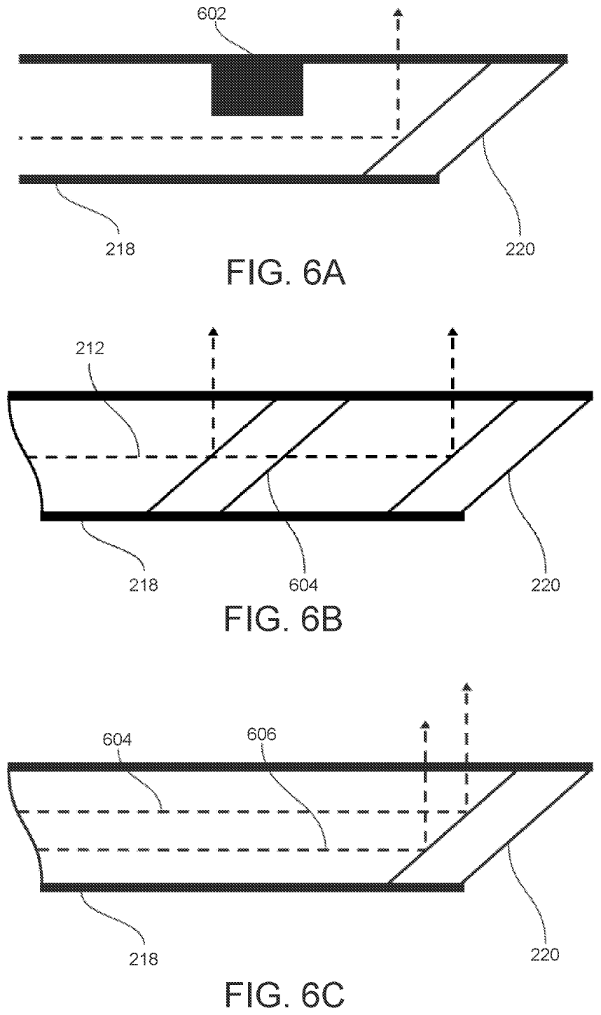

Referring to FIGS. 6A-6C, different non-limiting examples of tube 218 and optical element 220 configurations are shown. For example, FIG. 6A illustrates an ultrasound transducer 602 supported in the tube 218 in addition to the optical element 220 for reflecting light for radial optical scanning.

FIG. 6B shows an example embodiment whereby two optical elements 220 and 604 are arranged in series in the tube 218. Here, the light beam 212 passes through optical element 604 which may be a partially reflecting mirror. The light beam 212 splits into a reflected portion for radial scanning and a straight beam that reaches the second optical element 220 which may further reflect the light beam for additional radial scanning at a second point.

A non-limiting example in FIG. 6C shows two light beams 604 and 606 from two waveguides being transmitted toward a single optical element 220. Due to the angle of the reflecting surface, beams 604 and 606 are reflected at two different points. The gap between the two points along the length of the tube 218 may be controlled by varying the radial distance between the parallel light beams 604 and 606.

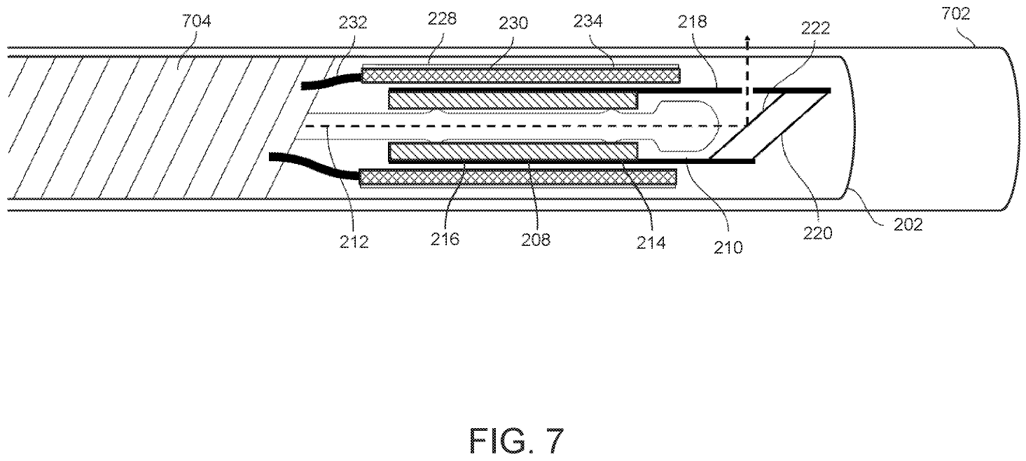

FIG. 7 illustrates a non-limiting example of the imaging assembly 200 enclosed in an outer sheath 702. The imaging assembly 200 may be longitudinally pulled back or retracted within an outer sheath for 3D volumetric measurement as the imaging assembly 200 rotates via the micromotor. The catheter sheath 202 may be a braided copper wire tube 704.

According to another exemplary embodiment of the present invention, the micromotor could be an electrostatic motor, which does not require a permanent magnet, but instead relies on the attraction and repulsion of electric charges to generation motion. Central to the disclosed invention, the waveguide may still function as the axle on which rotating components are supported, while the rotor and stator components may vary. The motor may also be driven by pneumatics such as in dental drills, or by a similar mechanism through hydraulics.

In general, any described force-generating mechanism may be driven by closed loop speed control with a wavelength-multiplexed optical channel and narrow-band reflecting grid. It may also be driven by open loop speed control provided by external driver electronics.

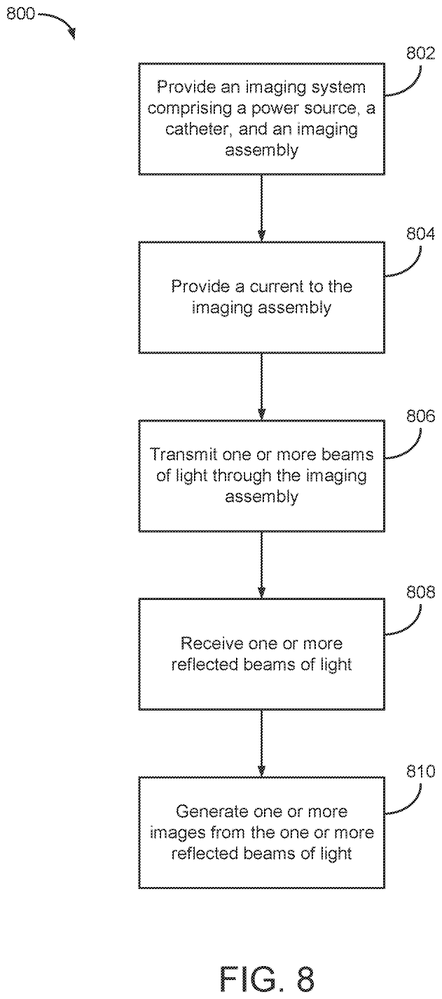

Referring to FIG. 8, a non-limiting method 800 for imaging using any of the above imaging system and imaging assembly configurations is described. At step 802, an imaging system is provided, such as the imaging systems described in FIG. 1. The imaging system includes an imaging assembly as described by any embodiment in FIGS. 2-7. At step 804, a current is supplied to the imaging assembly. Specifically, current is supplied to the electromagnetic coils to generate a magnetic field to cause the magnet to rotate. As described above, the magnet may be coupled to a tube and distal optical element, which will rotate with the magnet.

At step 806, one or more beams of light is transmitted through the imaging assembly via the waveguide. The light beam may travel through the distal tip of the waveguide, be reflected, diffracted, or refracted by the optical element for radial or forward scanning. At step 808, one or more reflected beams of light are received. For example, light may be reflected from tissue surrounding the catheter. Finally, as step 810, one more images are generated from the one or more reflected beams of light.

In a non-limiting example, steps 806-810 may utilize an ultrasound waveguide instead of an optical waveguide to transmit and receive ultrasound signal and generate an image therefrom.

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

What is claimed is:

1. An imaging system, comprising:
a power source;
a catheter having a proximal end and a distal end and defining a lumen therebetween; and
an imaging assembly including a proximal portion and a distal portion disposed within the lumen adjacent to the distal end of the catheter, the imaging assembly comprising:
a waveguide centered in the imaging assembly and extending a length of the catheter,
at least one permanent magnet positioned in the proximal portion of the imaging assembly and disposed radially around the waveguide,
an optical element coupled to an outer surface of the at least one permanent magnet, the optical element being disposed beyond the distal portion of the imaging assembly and into the distal end of the catheter, and
one or more electromagnetic coils positioned in the proximal portion of the imaging assembly radially outward from the at least one permanent magnet and in electrical communication with the power source.

2. The imaging system of claim 1, wherein the at least one permanent magnet comprises a ring magnet.

3. The imaging system of claim 1, wherein the waveguide comprises one or more optical fibers.

4. The imaging system of claim 1, wherein the waveguide includes one or more radial protrusions.

5. The imaging system of claim 4, wherein the one or more radial protrusions are formed in the waveguide.

6. The imaging system of claim 4, wherein the one or more radial protrusions comprise bearings radially positioned on the waveguide.

7. The imaging system of claim 4, wherein the one or more radial protrusions abut the at least one permanent magnet.

8. The imaging system of claim 1, wherein a distal tip of the waveguide comprises a lens.

9. The imaging system of claim 1, wherein the optical element includes one or more mirrors or lenses.

10. The imaging system of claim 1, further comprising an extension coupling the optical element to the outer surface of the one or more permanent magnet.

11. The imaging system of claim 10, wherein the extension includes an opening in at least a portion thereof.

12. The imaging system of claim 11, wherein the waveguide is configured to transmit light toward the one or more mirrors whereupon the light is reflected through the opening in the extension.

13. The imaging system of claim 10, further comprising a support bearing coupled to the extension and abutting a distal end of the one or more electromagnetic coils.

14. The imaging system of claim 10, further comprising an ultrasound transducer couped to the extension.

15. The imaging system of claim 10, wherein the extension is a tube.

16. The imaging system of claim 1, wherein the electromagnetic coils are in electrical communication with the power source via one or more wires.

17. The imaging system of claim 1, wherein the electromagnetic coils generate a magnetic field when a current is supplied by the power source.

18. The imaging system of claim 17, wherein the at least one permanent magnet and optical element rotate based on a strength and a frequency of the magnetic field.

19. The imaging system of claim 1, further comprising a back iron positioned radially between the one or more electromagnetic coils and an inner surface of the catheter in the proximal portion of the imaging assembly.

20. The imaging system of claim 1, further comprising a spring positioned proximal to the at least one permanent magnet in the imaging assembly and oriented such that the waveguide passes through the spring.

21. An imaging assembly including a proximal portion and a distal portion disposed within a distal end of a catheter, the imaging assembly further comprising:
a waveguide centered in the imaging assembly and extending a length of the catheter,
at least one permanent magnet positioned in the proximal portion of the imaging assembly and disposed radially around the waveguide,
an optical element coupled to an outer surface of the at least one permanent magnet, the optical element being disposed beyond the distal portion of the imaging assembly and into the distal end of the catheter, and
one or more electromagnetic coils positioned in the proximal portion of the imaging assembly radially outward from the at least one permanent magnet.

22. The imaging assembly of claim 21, wherein the at least one permanent magnet comprises a ring magnet.

23. The imaging assembly of claim 21, wherein the waveguide is an optical fiber.

24. The imaging assembly of claim 21, wherein the waveguide includes one or more radial protrusions.

25. The imaging assembly of claim 24, wherein the one or more radial protrusions are formed in the waveguide.

26. The imaging assembly of claim 24, wherein the one or more radial protrusions comprise bearings radially positioned on the waveguide.

27. The imaging assembly of claim 24, wherein the one or more radial protrusions abut the at least one permanent magnet.

28. The imaging assembly of claim 21, wherein a distal tip of the waveguide comprises a lens.

29. The imaging assembly of claim 21, wherein the optical element includes one or more mirrors or lenses.

30. The imaging assembly of claim 21, further comprising an extension coupling the optical element to the outer surface of the one or more permanent magnet.

31. The imaging assembly of claim 30, wherein the extension includes an opening in at least a portion thereof.

32. The imaging assembly of claim 31, wherein the waveguide is configured to transmit light toward the one or more mirrors whereupon the light is reflected through the opening in the extension.

33. The imaging assembly of claim 30, further comprising a support bearing coupled to the extension and abutting a distal end of the one or more electromagnetic coils.

34. The imaging assembly of claim 30, further comprising an ultrasound transducer couped to the extension.

35. The imaging assembly of claim 30, wherein the extension is a tube.

36. The imaging assembly of claim 21, wherein the electromagnetic coils generate a magnetic field when a current is supplied by a power source.

37. The imaging assembly of claim 36, wherein the at least one permanent magnet and optical element rotate based on a strength and a frequency of the magnetic field.

38. The imaging assembly of claim 21, further comprising a back iron positioned radially between the one or more electromagnetic coils and an inner surface of the catheter in the proximal portion of the imaging assembly.

39. The imaging assembly of claim 21, further comprising a spring positioned proximal to the at least one permanent magnet in the imaging assembly and oriented such that the waveguide to passes through the spring.

40. An imaging method, comprising:
providing an imaging system comprising:
a power source;
a catheter having a proximal end and a distal end and defining a lumen therebetween; and
an imaging assembly having a proximal portion and a distal portion disposed within the lumen adjacent to the distal end of the catheter, the imaging assembly comprising:
a waveguide centered in the imaging assembly and extending a length of the catheter,
at least one permanent magnet positioned in the proximal portion of the imaging assembly and disposed radially around the waveguide,
an optical element coupled to an outer surface of the at least one permanent magnet, the optical element being disposed beyond the distal portion of the imaging assembly and into the distal end of the catheter, and
one or more electromagnetic coils positioned in the proximal portion of the imaging assembly radially outward from the at least one permanent magnet, and in electrical communication with the power source:
providing a current to the one or more electromagnetic coils to generate a magnetic field;
transmitting one or more beams of light through the waveguide toward the optical element;
receiving one or more reflected beams of light from the optical element; and
generating an image from the one or more reflected beams using a processor.

41. The imaging method of claim 40, wherein the optical element includes one or more mirrors or lenses.

42. The imaging method of claim 40, further comprising an extension coupling the optical element to the outer surface of the one or more permanent magnet.

43. The imaging method of claim 42, wherein the extension includes an opening in at least a portion thereof.

44. The imaging method of claim 43, further comprising transmitting light using the waveguide toward the one or more mirrors such that the light is reflected through the opening in the extension.

45. The imaging method of claim 42, further comprising providing a support bearing coupled to the extension and abutting a distal end of the one or more electromagnetic coils.

46. The imaging method of claim 42, further comprising transmitting ultrasound energy using an ultrasound transducer couped to the extension.

47. The imaging method of claim 42, wherein the extension is a tube.

48. The imaging method of claim 40, further comprising electrically connecting the electromagnetic coils with the power source via one or more wires.

49. The imaging method of claim 48, further comprising generating a magnetic field using the electromagnetic coils by transmitting a current from the power source.

50. The imaging method of claim 49, further comprising rotating the at least one permanent magnet and optical element based on a strength and a frequency of the magnetic field.

51. The imaging method of claim 40, further comprising providing a back iron positioned radially between the one or more electromagnetic coils and an inner surface of the catheter in the proximal portion of the imaging assembly.

52. The imaging method of claim 40, further comprising providing a spring positioned proximal to the at least one permanent magnet in the imaging assembly and oriented such that the waveguide passes through the spring.
